(19) United States
(12) Patent Application Publication
(10) Pub. No.: US 2010/0324369 A1
(43) Pub. Date: Dec. 23, 2010

(54) SURGICAL OPTICAL ACCESS APPARATUS

(76) Inventor: Robert C. Smith, Middletown, CT (US)

Correspondence Address: Tyco Healthcare Group LP;d/b/a Covidien, 555 Long Wharf Drive, Mail Stop 8-N1, Legal Department, New Haven, CT, 06511, US

(21) Appl. No.: 12/526,081

(22) PCT Filed: Feb. 26, 2008
(86) PCT No.: PCT/US08/02459

§ 371 Date: Aug. 6, 2009

Related U.S. Application Data
(60) Provisional application No. 60/904,336, filed on Feb. 28, 2007.

(51) Int. Cl. A61B 1/313; A61B 17/34

(52) U.S. Cl. 600/138; 606/185

(57) ABSTRACT

An optical access apparatus includes a portal sleeve defining a longitudinal axis and a longitudinal opening therethrough and an inner member at least partially positionable within the portal sleeve. The portal sleeve has leading and trailing end portions. The leading end portion has a leading edge adapted to penetrate tissue. The inner member includes a longitudinal lumen for at least partial reception of a viewing device and has a generally closed optical window adapted to permit passage of an image for viewing by the viewing device. The closed optical window is generally atraumatic with respect to the tissue whereby penetration through the tissue is substantially effected through the leading edge of the portal sleeve.

### CROSS-REFERENCE TO RELATED APPLICATION

The present application claims the benefit of and priority to U.S. Provisional Application Ser. No. 60/904,336, filed on Feb. 28, 2007, the entire disclosure of which is incorporated herein by reference.

### BACKGROUND

1. Technical Field

The present disclosure relates to an apparatus for penetrating body tissue during minimally invasive surgical procedures, such as endoscopic or laparoscopic procedures. More particularly, the present disclosure relates to an access assembly having a transparent window for providing visual observation during penetration of the peritoneum or other body tissue.

2. Background of the Related Art

Minimally invasive surgical procedures including endoscopic and laparoscopic procedures permit surgery to be performed on organs, tissue and vessels far removed from an opening within the tissue. Laparoscopic and endoscopic procedures are performed in the interior of the abdomen through a small incision such as, for example, a narrow endoscopic tube or cannula inserted through a small entrance incision in the skin. Typically, after the abdominal cavity is insufflated, a trocar is used to puncture the cavity wall, i.e., the peritoneal lining, to create a pathway to the underlying surgical site. Generally, the trocar includes a stylet or an obturator having a sharp tip for penetrating the body cavity, which is positioned coaxially within an outer cannula. The obturator is removed, leaving the outer cannula in place for reception of instrumentation utilized to perform the surgical procedure. An example of a known trocar is described in commonly assigned U.S. Pat. No. 6,319,266 to Stellon, which issued Nov. 21, 2001, the contents of which are incorporated herein in its entirety by reference. However, with known trocars, advancement of the obturator through tissue is typically performed blind, i.e., without visualization of the tissue being entered. Obturators allowing visualization include U.S. Pat. Nos. 5,334,150, 5,431,151 and 5,441,041.

Accordingly, the present disclosure provides an optical access assembly which permits direct visualization of body tissue during penetration of the body cavity. Moreover, the optical access assembly of the present disclosure provides an improved structure for direct visualization of the body tissue being penetrated and serves as a conduit for subsequent introduction of surgical instrumentation required for performance of the surgical procedure.

### SUMMARY

An optical access apparatus includes a portal sleeve defining a longitudinal axis and a longitudinal opening therethrough and an inner member at least partially positionable within the portal sleeve. The portal sleeve has leading and trailing end portions. The leading end portion has a leading edge adapted to penetrate tissue. The inner member includes a longitudinal lumen for at least partial reception of a viewing device and has a generally closed optical window adapted to permit passage of an image for viewing by the viewing device. The closed optical window is generally atraumatic with respect to the tissue whereby penetration through the tissue is substantially effected through the leading edge of the portal sleeve.

The leading edge of the portal sleeve may define a sharp edge adapted to pierce through tissue. Preferably, the leading edge defines a forward most edge surface which is longitudinally spaced a predetermined distance relative to the closed optical window. The leading edge also may include an oblique edge surface extending from the forward most edge surface and being obliquely arranged with respect to the longitudinal axis. The oblique edge surface may be generally arcuate and may define a general concave arrangement.

The optical window defines a leading end having an oblique surface obliquely arranged with respect to the longitudinal axis. The oblique surface defines a general arcuate characteristic and generally follows the profile of the leading edge of the portal sleeve. The oblique surface of the leading end defines a general concave characteristic.

The inner member may include means to rotationally align the inner member with respect to the portal sleeve to ensure that the leading end of the optical window is aligned with the leading edge of the portal sleeve. A base may be connected to the inner member and a portal housing connected to the portal sleeve. The base and the portal housing have structure to rotationally fix the inner member relative to the portal sleeve.

### BRIEF DESCRIPTION OF THE DRAWINGS

Preferred embodiments of the present disclosure are described hereinbelow with references to the drawings, wherein:

FIG. 1 is a perspective view of an optical access apparatus constructed in accordance with the principles of the present disclosure illustrating the outer portal and the inner obturator positioned within the outer portal;

FIG. 2 is an enlarged isolated view of the leading end of the outer portal;

FIG. 3 is side cross-sectional view of the optical access apparatus taken along the lines 3-3 of FIG. 1;

FIG. 4 is an enlarged isolated view of the area of detail indicated in FIG. 3; and

FIG. 5 is a side cross-sectional view illustrating insertion of the optical access apparatus through tissue under visualization provided by a viewing device introduced within the optical access apparatus.

### DETAIL DESCRIPTION OF PREFERRED EMBODIMENTS

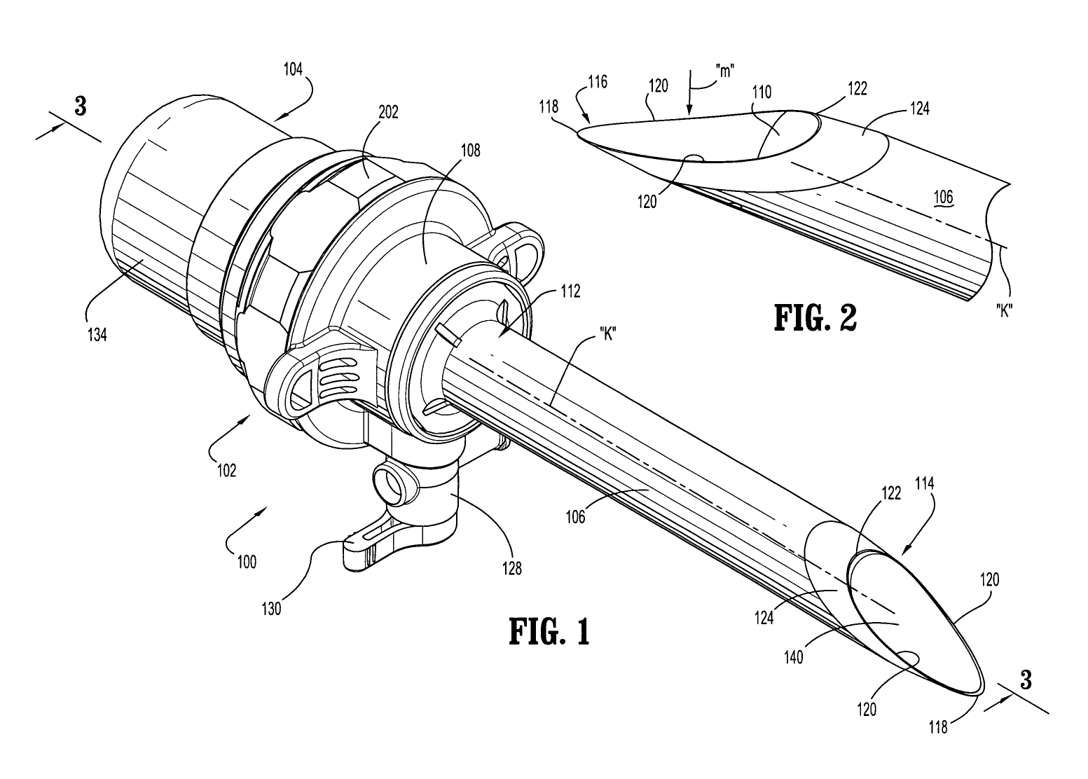

Referring now in detail to the drawing figures, in which, like reference numerals identify similar or identical elements, there is illustrated in FIG. 1, an optical access apparatus 100 constructed in accordance with a preferred embodiment of the present disclosure, and designated generally by reference numeral 100. Optical access apparatus 100 contemplates the direct visualization of body tissue during penetration of the peritoneal cavity or other tissue portions. In addition, optical access apparatus 100 facilitates the introduction of various types of surgical instruments such as, for example, an endoscopic clip applier, grasper, dissector, retractor, stapler, photographic device, tube, and the like. Optical access apparatus 100 is dimensioned to pass through body tissue and may incorporate structure to cut, puncture, or pierce the body tissue.

Generally, optical access apparatus 100 includes outer portal member 102 and inner member 104 which is at least partially positionable within the outer portal member 102. In one embodiment, optical access apparatus 100 is a laparoscopic trocar assembly particularly adapted for use in laparoscopic surgery where the peritoneal cavity is insufflated with a suitable gas, e.g., CO₂, to raise the cavity wall from the internal organs therein. Specifically, outer portal member 102 with inner member 104 positioned therein is applied against the body cavity or abdominal wall. Once optical access apparatus 100 penetrates through the abdominal wall, inner member 104 is removed from outer portal member 102 to permit introduction of surgical instrumentation through the remaining outer portal member 102 to perform a surgical procedure. In one embodiment, penetration and advancement of optical access apparatus 100 is performed under visualization by way of an imaging device, e.g., or endoscope, positioned within inner member 104 as will be discussed.

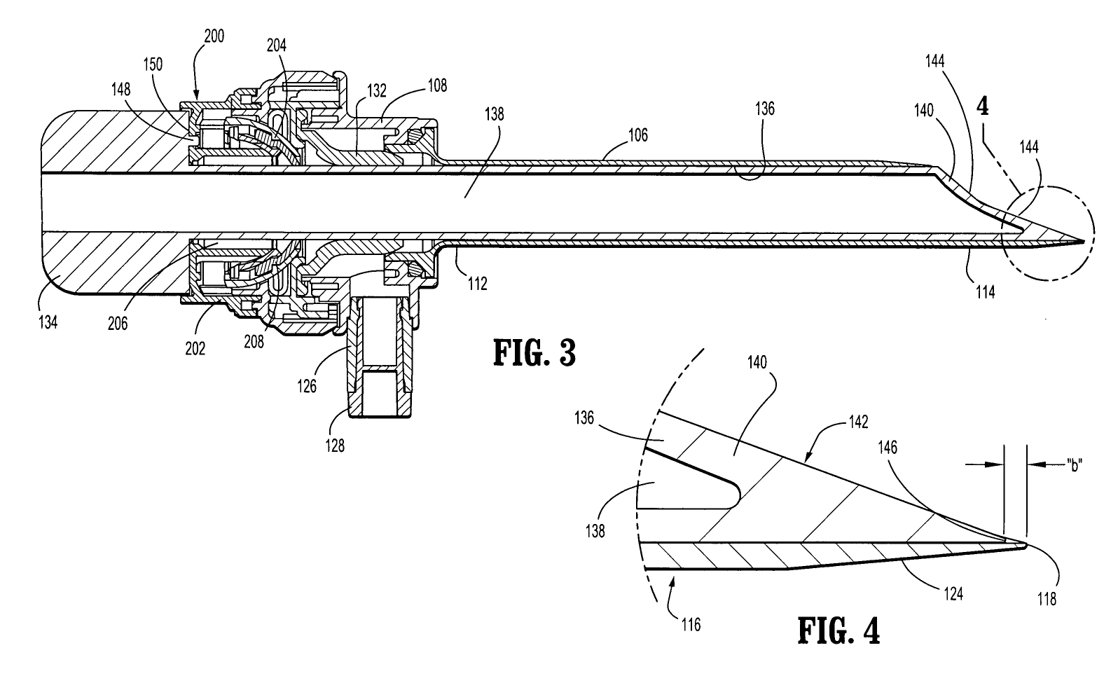

Referring now to FIGS. 1-2, in conjunction with FIG. 3, outer portal member 102 includes portal sleeve 106 and portal housing 108 mounted to an end of the portal sleeve 106. Any means for mounting portal sleeve 106 to portal housing 108 are envisioned including threaded arrangements, bayonet coupling, snap-fit arrangements, adhesives, etc. As an alternative, portal sleeve 106 and portal housing 108 may be integrally or monolithically formed. Portal sleeve 106 defines longitudinal axis “k” extending along the length of the portal sleeve 106. Portal sleeve 106 and portal housing 108 further defines internal longitudinal passage 110 dimensioned to permit passage of surgical instrumentation, e.g., such as inner member 104 or, in the absence of the inner member 104 and when portal sleeve 106 is positioned to access tissue, surgical instruments such as surgical forceps, dissectors, graspers, staplers, etc. required to perform the desired procedure. Portal sleeve 106 may be formed of stainless steel or other rigid materials such as a polymeric material or the like. Portal sleeve 106 may be clear or opaque. The diameter of portal sleeve 106 may vary, but, typically ranges from about 10 mm to about 15 mm.

Portal sleeve 106 defines proximal or trailing end portion 112 and distal or leading end portion 114. Leading end portion 114 defines generally annular leading edge 116. Leading edge 116 includes outermost edge surface 118, oblique edge surfaces 120 extending contiguously from the outermost edge surface 118 and innermost or trailing edge surface 122 extending contiguously from the oblique edge surfaces 120. Outermost edge surface 118 is substantially arcuate as shown defining a relatively large radius of curvature “m”. Oblique edge surfaces 120 are each generally arranged in oblique relation to the longitudinal axis “k”. In one embodiment, each oblique edge surface 120 defines a curved, radiussed or arcuate concave profile. This curved profile provides a “hollow ground” appearance to leading edge 116. Such “hollow ground” arrangement provides enhanced ability to penetrate tissue. In one embodiment, leading edge 116 may be sufficiently sharp to facilitate piercing of tissue. Alternatively, leading edge 116 may be atraumatic or blunt. Leading edge 116 in combination with the “hollow ground” arrangement of oblique edge surfaces 120 provides the primary penetrating function of optical access apparatus 100.

With reference to FIGS. 3-4, in combination with FIGS. 1-2, leading end portion 114 further defines tapered section 124 extending contiguously from leading edge 116 to the main body of portal sleeve 106. Tapered section 124 defines an outer diameter or dimension which gradually increases from distal to proximal to define the tapered, e.g., sharpened arrangement, to thereby facilitate continued advancement of portal sleeve 106 into the tissue. In one embodiment, tapered portion 124 also forms the sharpened aspect of leading edge 116.

Portal housing 108 includes port opening 126 and luer fitting 128 positioned within the port opening 126. Luer fitting 128 is adapted for connection to a supply of insufflation gases as is conventional in the art and incorporates valve 130 (FIG. 1) to selectively open and close the passage of the luer fitting 128. Portal housing 108 further includes internal duckbill or zero closure valve 132 which is adapted to open to permit passage of the surgical instrumentation and closes in the absence of the instrumentation. Closure valve 132 is preferably adapted to close upon exposure to the forces exerted by the insufflation gases in the internal cavity. Other zero closure valves are also contemplated including single or multiple slit valve arrangements, trumpet valves, flapper valves, etc.

Portal apparatus 100 also may incorporate seal assembly 200. Seal assembly 200 may be a separate component from outer portal member 102 and, accordingly, adapted for releasable connection to the portal housing 104. Alternatively, seal assembly 200 may be incorporated as part of outer portal member 102. Seal assembly 200 includes seal housing, generally identified as reference numeral 202, and gimbal mount 204 which is disposed within the seal housing 202. Seal housing 202 houses the sealing components of the assembly and defines the outer valve or seal body of the seal assembly 200. Seal housing 202 may incorporate multiple housing components, or may be a single unit. Seal housing 202 defines central passage 206 which is dimensioned to receive a surgical instrument and laterally confine the instrument within seal housing 202.

Gimbal mount 204 is mounted in a manner to permit angulation and/or rotational movement of the gimbal mount 204 relative to, or about, longitudinal axis “k”. Specifically, gimbal mount 204 is free to angulate relative to longitudinal axis “k” through a range of motion within seal housing 202. Gimbal mount 204 incorporates seal 208 which is adapted to form a substantial sealing relation about an instrument positioned within seal housing 202. Further details of gimbal mount 204 may be ascertained by reference to commonly assigned U.S. Patent Publication No. 2006/0224120 to Smith, the entire contents of which are incorporated herein by reference.

With reference to FIGS. 3-4, in conjunction with FIG. 1, inner member 104 of optical access apparatus 100 includes base 134 and elongate member 136 extending from the base 134. Base 134 is generally circular in cross-section and is dimensioned for engagement by the user. Base 134 and elongate member 136 defines longitudinal passage 138 for receiving a viewing device. Elongate member 136 further define closed optical window 140. Optical window 140 defines a side profile which generally corresponds with the profile of leading end portion 114 of outer portal member 102. In particular, optical window 140 has a general arcuate or recessed face 142 which is obliquely arranged with respect to the longitudinal axis “k”. Recessed face 142 may be curved or, in one embodiment, may include intersecting planar face portions 144 arranged to define the arcuate configuration shown. Recessed face 142 includes atraumatic tip 146. Atraumatic tip 146 is generally arcuate or blunted as shown such that the tip 146 is not capable of incising or piercing tissue. Atraumatic tip 146 is preferably spaced a predetermined distance “b” from outermost edge surface 118 of portal sleeve 106. This arrangement, e.g., permits leading edge 116 of portal sleeve 106 to initially engage the tissue to perform the primary penetrating function of optical access apparatus.

Optical window 140 is intended to transfer light or an image therethrough and may or may not have refracting characteristics. Optical window 140 permits visualization during penetration of the body tissue. Optical window 140 may comprise a transparent or translucent polymeric material and be fabricated via known injection molding techniques. Alternatively, window 140 may comprise an optical glass. The term “transparent” is to be interpreted as having the ability to permit the passage of light with or without clear imaging capabilities. Moreover, the transparent material includes any material which is not opaque. It is also to be appreciated that only a portion of optical window 140 may be transparent. Thus, a portion of, or the entire optical window 140, may be transparent or translucent. Optical window 140 may have a unitary construction or be comprised of multiple parts.

Window 140 may include an image directing member (not shown) for directing optical images into longitudinal passage 138 of inner member 104 or back to an imaging device positioned within the inner member 104. The image directing member may be a lens, an optical prism, an optical mirror, or like image directing medium.

As best depicted in FIG. 3, inner member 104 may also include a mechanism for ensuring that the inner member 104 is at a desired rotational arrangement relative to outer portal member 102 thereby ensuring that optical window 140 of the inner member 104 and leading edge 116 of the outer portal member 102 are in the aligned arrangement depicted in FIG. 3. In one embodiment, the mechanism includes an alignment pin 148 extending from base 134 of inner member 104. Alignment pin 148 is received within a corresponding alignment opening 150 of portal housing 108 to rotatably fix the inner member 104 relative to outer portal member 102.

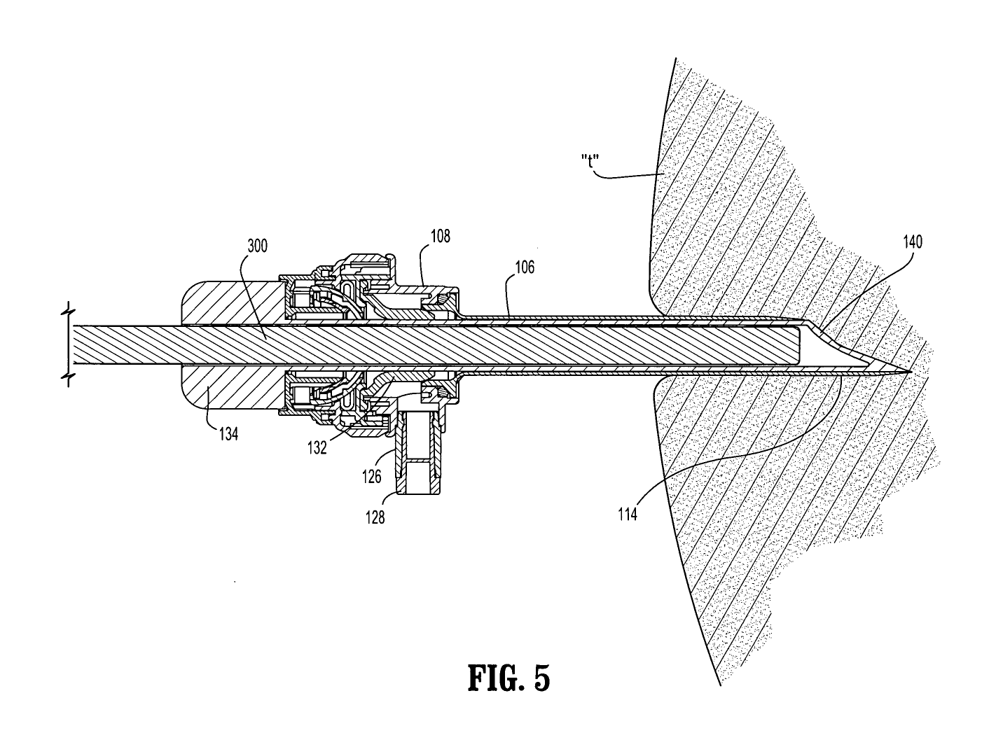

In operation, the peritoneal cavity is insufflated to raise the cavity wall to provide greater access to tissue and organs therewithin. An endoscope 300 is inserted into optical access assembly 100, i.e., through obturator base 134, seal housing 202, portal housing 102 and into elongate member 136 of inner member 104 as shown in FIG. 5. One suitable endoscope for use with optical access assembly 100 is disclosed in commonly assigned U.S. Pat. No. 5,718,664 to Peck et al., the contents of which are incorporated herein by reference. Instrument seal 208 of gimbal mount 204 of seal housing 202 forms a fluid tight seal about endoscope 300. As appreciated, endoscope 300 is advanced within inner member 104 until the distal end of the endoscope 300 is adjacent optical window 140. In this position, the distal lens element of the endoscope 300 is capable of viewing the tissue to be penetrated by optical access apparatus 100. Endoscope 300 may be secured relative to optical access apparatus 100 with a locking system (not shown), at the proximal end of the optical access assembly 100, at some location along portal sleeve 104, or at the distal end of the portal sleeve 104. One suitable locking mechanism may include a cam or wedge mechanism mounted adjacent portal housing 102 and adapted to engage the outer surface of endoscope 300. Other locking mechanisms are also envisioned.

The procedure is continued by positioning optical window 140 against the body tissue “t” and advancing the optical access apparatus 100 within the tissue. A skin incision may be made before pressing access apparatus 100 against the tissue, if desired. During penetration within tissue, optical window 140 serves substantially no piercing function. Rather, the primary piercing capabilities are performed by leading edge 116 of portal sleeve 106. The hollow ground appearance of leading edge 116 in combination with the matching profile of optical window 140 facilitates entry and passage through tissue as discussed hereinabove. The surgeon observes with endoscope 300 the underlying tissue during penetration to ensure there is no undesired contact with organs, tissue, etc. lying beneath the peritoneal lining. In instances where a video system is utilized, the surgeon simply observes the penetration of body tissue “t” via any known video monitor. Once the surgeon penetrates the body tissue “t” as observed through the endoscope 300, the surgeon discontinues the application of force.

After penetration into the underlying body cavity, inner member 104 and endoscope 300 are removed. Surgical instrumentation may be introduced within outer portal member 102 to perform the desired procedure.

It will be understood that various modifications can be made to the embodiments of the present invention herein disclosed without departing from the spirit and scope thereof. For example, various diameters for the obturator assembly, cannula assembly, as well as various diameters for the surgical instruments are contemplated. Also, various modifications may be made in the configuration of the parts. Therefore, the above description should not be construed as limiting the invention but merely as exemplifications of preferred embodiments thereof. Those skilled in the art will envision other modifications within the scope and spirit of the present invention as defined by the claims appended hereto.

What is claimed is:

1. An optical access apparatus, which comprises:
a portal sleeve defining a longitudinal axis and a longitudinal opening therethrough, the portal sleeve having leading and trailing end portions, the leading end portion having a leading edge adapted to penetrate tissue; and
an inner member at least partially positionable within the portal sleeve, the inner member including a longitudinal lumen for at least partial reception of a viewing device and having a generally closed optical window adapted to permit passage of an image for viewing by the viewing device, the closed optical window being generally atraumatic with respect to the tissue whereby penetration through the tissue is substantially effected through the leading edge of the portal sleeve.

2. The optical access apparatus according to claim 1 wherein the leading edge of the portal sleeve defines a sharp edge adapted to pierce through tissue.

3. The optical access apparatus according to claim 1 wherein the leading edge of the portal sleeve includes a forwardmost edge surface which is longitudinally spaced a predetermined distance relative to the closed optical window.

4. The optical access apparatus according to claim 1 wherein the leading edge includes an oblique edge surface, the oblique edge surface extending from the forwardmost edge surface and being obliquely arranged with respect to the longitudinal axis.

5. The optical access apparatus according to claim 4 wherein the oblique edge surface is generally arcuate.

6. The optical access apparatus according to claim 5 wherein the oblique edge surfaces defines a general concave arrangement.

7. The optical access apparatus according to claim 4 wherein the closed optical window defines a leading end obliquely arranged with respect to the longitudinal axis.

8. The optical access apparatus according to claim 7 wherein the leading end of the optical window defines a general arcuate characteristic.

9. The optical access apparatus according to claim 8 wherein the leading end of the optical window defines a general concave characteristic.

10. The optical access apparatus according to claim 1 wherein the inner member includes means to rotationally align the inner member with respect to the portal sleeve.

11. The optical access apparatus according to claim 1 including a base connected to the inner member and a portal housing connected to the portal sleeve, the base and the portal housing having structure to rotationally fix the inner member relative to the portal sleeve.
